(19) United States
(12) Patent Application Publication
(10) Pub. No.: US 2026/0028315 A1
(43) Pub. Date: Jan. 29, 2026

(54) IMPROVED PREPARATION METHOD FOR RUCAPARIB

(71) Applicant: KOREA UNIVERSITY RESEARCH AND BUSINESS FOUNDATION, Seoul (KR)

(72) Inventors: Cheol-Hong CHEON, Seoul (KR); Jinjae PARK, Seoul (KR)

(73) Assignee: KOREA UNIVERSITY RESEARCH AND BUSINESS FOUNDATION, Seoul (KR)

(21) Appl. No.: 18/843,393

(22) PCT Filed: Feb. 27, 2023
(86) PCT No.: PCT/KR2023/002732

§ 371 Date: Mar. 6, 2025

(30) Foreign Application Priority Data
Mar. 2, 2022 (KR) 10-2022-0026717

(51) Int. Cl. C07D 209/14; C07D 487/06

(52) U.S. Cl. CPC C07D 209/14; C07D 487/06

(57) ABSTRACT

The present disclosure relates to an improved method for preparing rucaparib capable of achieving excellent synthesis yield and reproducibility. In particular, the present disclosure relates to an improved method for synthesizing Rucaparib and a novel intermediate that can be used in its preparation. The method offers the advantage of using of 4-cyanobenzaldehyde in the synthesis of an indole skeleton with substituents introduced at the 2, 3, 4, and 6 positions and then, simultaneously reducing the cyano group to introduce both a lactam and an amine group, and also relates to a novel intermediate that can be used in its preparation.

### FIELD OF THE INVENTION

The present disclosure relates to an improved method for preparing rucaparib capable of achieving excellent synthesis yield and reproducibility. In particular, the present disclosure relates to an improved method for synthesizing Rucaparib and a novel intermediate that can be used in its preparation. The method offers the advantage of using of 4-cyanobenzaldehyde in the synthesis of an indole skeleton with substituents introduced at the 2, 3, 4, and 6 positions and then, simultaneously reducing the cyano group to introduce both a lactam and an amine group, and also relates to a novel intermediate that can be used in its preparation.

### BACKGROUND OF THE INVENTION

Rucaparib (trade name: rubraca) is an anticancer drug approved as a treatment for ovarian cancer through poly(ADP-ribose) polymerase (PARP) inhibition in late 2016 by the US FDA. After the approval by the US FDA in 2016, rucaparib was approved for use in Europe as well in 2017, and has been used as a treatment for ovarian cancer through PARP inhibition in the US and five European countries (UK, Germany, France, Italy and Spain) since 2018. In addition to ovarian cancer, rucaparib was also approved for prostate cancer in 2020, and is in extensive preclinical stages for many other types of cancer, including breast cancer.

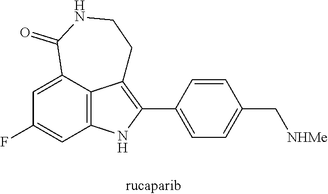 [SMILES: CNCc1ccc(-c2[nH]c3cc(F)cc4c3c2CCNC4=O)cc1]

Currently, rucaparib is mass produced through a synthetic route (Scheme 1) developed by Pfizer Inc. in 2012, however, this synthetic route proceeds through a linear sequence, and in some steps, synthesis yield is somewhat low and there is a problem with reproducibility. In particular, considering the marketability of rucaparib as a treatment for ovarian cancer and that possibility of rucaparib as a targeted anticancer drug for other types of cancer using a similar anticancer mechanism is investigated, development of a novel synthetic method for a rucaparib compound is necessary.

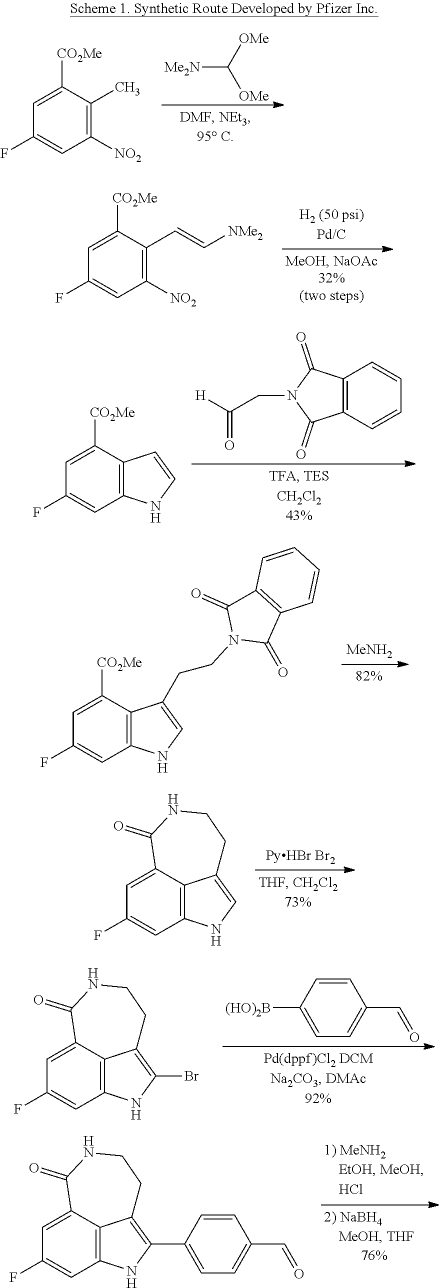

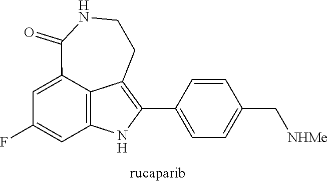 [SMILES: CNCc1ccc(-c2[nH]c3cc(F)cc4c3c2CCNC4=O)cc1]

Recognizing the need for developing such a novel synthetic method, numerus patents and papers on novel synthetic methods of this anticancer drug have been published after the FDA approval in 2016, however, most of them have focused on the synthesis of indoloazepine compound, a key intermediate in the existing synthetic method, and synthetic methods significantly improving the synthetic route have not been developed.

In view of the above, the inventors of the present disclosure have found a novel synthetic method that synthesizes rucaparib by, unlike the previous synthetic route, first synthesizing an indole skeleton to which substituents are introduced to positions 2, 3, 4 and 6, and then performing a heptagonal lactam ring formation reaction between the substituents introduced to positions 3 and 4, and have completed the present invention, in particular, by using of 4-cyanobenzaldehyde in the synthesis of an indole skeleton with substituents introduced at the 2, 3, 4, and 6 positions and then, simultaneously reducing the cyano group to introduce both a lactam and an amine group. In this case, the present invention is capable of achieving excellent synthesis yield and reproducibility in manufacturing steps.

### DETAILED DESCRIPTION OF THE INVENTION

### Technical Problem

The objective of the present disclosure is to provide an improved method for preparing rucaparib capable of achieving excellent synthesis yield and reproducibility.

Another objective of the present disclosure is to provide a novel intermediate that can be used in preparation of rucaparib.

### Technical Solution

To achieve the above objectives, one embodiment of the present disclosure provides a method for preparing a compound of Chemical Formula (3), the method including: reacting a compound of the following Chemical Formula (1) and a compound of the following Chemical Formula (2); and converting the result into a compound of the following Chemical Formula (3) in the presence of a catalyst:

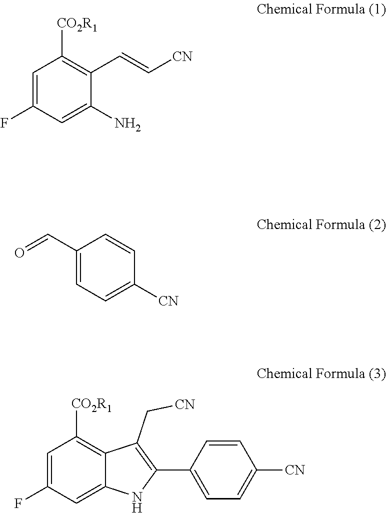

Herein,
R₁ is a linear or branched C₁-C₈alkyl.

The term “C₁-C₈alkyl” used in the present disclosure means a hydrocarbon having 1 to 5 carbon atoms, and the term “linear or branched” means that the hydrocarbon includes normal, secondary or tertiary carbon atoms. Specifically, proper examples of the “C₁-C₈alkyl” include methyl, ethyl, 1-propyl(n-propyl), 2-propyl, 1-butyl, 2-methyl-1-propyl, 3-pentyl and the like, but are not limited thereto.

In one embodiment of the present disclosure, the step of conversion into the compound of Chemical Formula (3) is preferably performed in the presence of a dehydrating agent. Using such a dehydrating agent may facilitate the overall reaction by removing water molecules generated when forming an imine intermediate.

In one embodiment of the present disclosure, preferred examples of the dehydrating agent include at least one compound selected from the group consisting of TiCl₄, MgSO₄ and Na₂SO₄, but are not limited thereto. In addition, in one embodiment of the present disclosure, the step of conversion into the compound of Chemical Formula (3) may be performed by reacting with molecular sieves, or using an azeotropic distillation.

In one preferred embodiment of the present disclosure, the catalyst used in the step of conversion into the compound of Chemical Formula (3) is MCN or N-heterocyclic carbene, and herein, M is an alkali metal or NR₄⁺, and R is H or a linear or branched C₁-C₈alkyl. The catalyst performs a role of facilitating a reaction in which an imine intermediate produced in the middle of the reaction forms an indole skeleton through cyclization.

In a preferred embodiment of the present disclosure, preferred examples of the N-heterocyclic carbene includes a compound selected from the group consisting of imidazolium, triazolium and thiazolium, but are not limited thereto.

Also, in one embodiment of the present disclosure, a method for preparing rucaparib represented by the following compound 1 includes:
(a) reacting a compound of the following Chemical Formula (1) and a compound of the following Chemical Formula (2), and converting the result into a compound of the following Chemical Formula (3) in the presence of a catalyst;
(b) performing a reduction reaction of the compound of Chemical Formula (3) and protecting the obtained primary amine with P1 and P2, thereby converting it into a compound of Chemical Formula (4);
(c) performing a deprotection reaction on the compound of Chemical Formula (4), followed by or simultaneously with a lactam ring formation reaction to obtain a compound of Chemical Formula (5); and
(d) performing a monomethylation reaction on the primary amine group of the compound of Chemical Formula (5) to produce Compound 1.

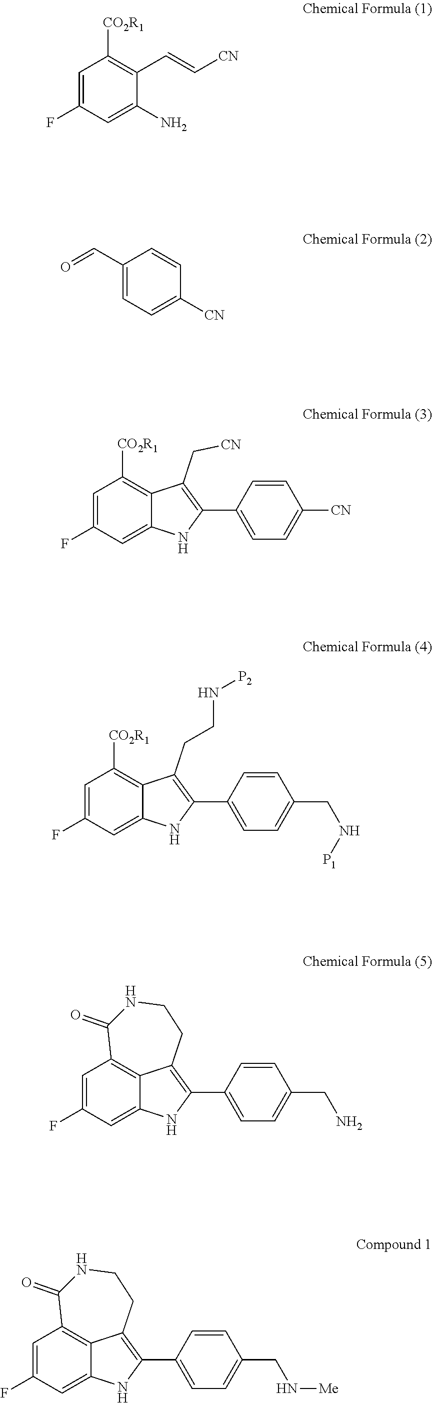

herein,
R is a linear or branched C₁-C₅alkyl; and

P₁ and P₂ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

The term “protecting group” used in the present disclosure refers to a moiety of a compound completely shielding or altering properties of a functional group or properties of the compound. Chemical substructures of a protecting group are very diverse. One function of a protecting group is to act as an intermediate in the synthesis of a parent drug substance. Chemical protecting groups and strategies for protection/deprotection are widely known in the related art. Regarding this, literatures [“Protective Groups in Organic Chemistry”, Theodora W. Greene (John Wiley & Sons, Inc., New York, 1991)] and [Protective Groups in Organic Chemistry, Peter G. M. Wuts and Theodora W. Greene, 4th Ed., 2006] are referenced. Protecting groups are often used to shield reactivity of a particular functional group and assist in efficiency of a target chemical reaction. Protection of a functional group of a compound alters other physical properties other than reactivity of the protected functional group, such as polarity, hydrophobicity, hydrophilicity, and other properties that may be measured using common analytical tools. Chemically protected intermediates may be biologically and chemically active or inactive as they are. The term “amine protecting group” refers to a protecting group useful for protecting an amine group (—NH2).

In a preferred embodiment of the present disclosure, preferred examples of the “amine protecting group” include methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc), however, the amine protecting group is not limited thereto, and protecting groups that may perform a role chemically equivalent to the protecting group are included in the category of the present disclosure.

In one embodiment of the present disclosure, the reduction reaction in step (b) is preferably performed in the presence of nickel boride.

In one embodiment of the present disclosure, the reduction reaction in step (b) is preferably a hydrogenation reaction performed in the presence of a metal catalyst selected from the group consisting of Ni, Pd, and Pt.

In one embodiment of the present disclosure, the reduction reaction in step (b) is preferably performed in the presence of a metal catalyst selected from the group consisting of Ni, Zn, Fe and Co, and a silane compound, or performed in the presence of a metal hydride selected from the group consisting of DIBAL-H, L-selectride, NaBH₄ and borane.

In a preferred embodiment of the present disclosure, examples of the silane compound used in the present disclosure include PhSiH₃, Ph₂SiH₂, Ph₃SiH, (EtO)₃SiH, Et₃SiH, MezSiHSiHMe₂, PMHS (polymethylhydrosiloxane), TMDS (1,1,3,3-tetramethyldisiloxane) and the like, but are not limited thereto.

In one embodiment of the present disclosure, the deprotection reaction in step (c) is performed under acidic conditions, with preferred acids being TFA or HCl.

In one embodiment of the present disclosure, the lactam ring formation reaction in step (c) is performed under basic conditions. Various inorganic and organic bases can be used to provide the basic conditions, with inorganic bases being preferred. Examples of inorganic bases that can be used in the present disclosure include sodium acetate, sodium carbonate, sodium bicarbonate, sodium phosphate, sodium hydroxide, lithium acetate, lithium carbonate, lithium bicarbonate, lithium phosphate, lithium hydroxide, potassium acetate, potassium carbonate, potassium bicarbonate, potassium phosphate, potassium hydroxide, calcium carbonate, calcium bicarbonate, calcium hydroxide, barium carbonate, and barium hydroxide, but are not limited to these.

In one embodiment of the present disclosure, the inorganic base used in the lactam ring formation reaction in step (c) is preferably an alkali metal hydroxide such as lithium, sodium, or potassium hydroxide. The most preferred alkali metal hydroxide is sodium hydroxide.

In one embodiment of the present disclosure, the monomethylation reaction in step (d) can be performed in the presence of various methyl precursors, catalysts, and solvents for the selective monomethylation reaction of a general primary amine. Specifically, the monomethylation of can be performed by reacting the primary amine in the compound of Chemical Formula (5) with Me-X (where X is a halogen or a corresponding leaving group) in a one-step reaction. In one preferred embodiment of the present disclosure, examples of Me-X compounds used in the present disclosure include iodomethane, but are not limited thereto.

Additionally, in one embodiment of the present disclosure, the monomethylation reaction in step (d) can be performed by reacting the primary amine of the compound of Chemical Formula (5) with CHXYZ (where X is a halogen or a corresponding leaving group, and Y and Z are substituents that can be replaced with hydrogen) to introduce a carbon atom, followed by replacing Y and Z with hydrogen in a multi-step process. Alternatively, the monomethylation reaction in step (d) can be performed by reacting the primary amine of Chemical Formula (5) with CH₂XY (where X is a halogen or a corresponding leaving group, and Y is a substituent that can be replaced with hydrogen, such as TMS) to introduce a CH₂Y group, followed by replacing Y with hydrogen in a multi-step process. In one preferred embodiment of the present disclosure, examples of CH₂XY compounds used include (iodomethyl)trimethylsilane, but are not limited thereto.

Additionally, in one embodiment of the present disclosure, a novel intermediate compound that can be used in its preparation is provided, represented by the following the compound of Chemical Formula (3):

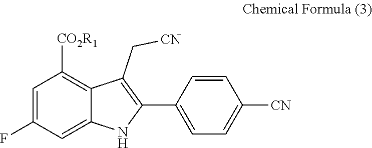

herein,
R₁ is a linear or branched C₁-C₅ alkyl.

Any suitable solvent can be used in the method of the present disclosure. Representative solvents include pentane, pentanes, hexane, hexanes, heptane, heptanes, petroleum ether, cyclopentane, cyclohexane, benzene, toluene, xylene, dichloromethane, trifluoromethylbenzene, halobenzene such as chlorobenzene, fluorobenzene, dichlorobenzene and difluorobenzene, methylene chloride, chloroform, acetone, ethyl acetate, diethyl ether, tetrahydrofuran (THF), 2-methyltetrahydrofuran, dibutyl ether, diisopropyl ether, methyl tert-butyl ether, dimethoxyethane, dioxane (1,4 dioxane), N-methyl pyrrolidinone (NMP), DMF, alcohol such as methanol, ethanol, propanol and butanol, or mixtures thereof, but are not limited thereto.

The reaction mixture of each step of the present disclosure may be under any suitable pressure. For example, the reaction mixture may be under atmospheric pressure. The reaction mixture may also be exposed to any suitable environment such as atmospheric gas, or inert gas such as nitrogen or argon.

The reaction of each step of the present disclosure may be performed at any suitable temperature. For example, the temperature of the mixture during the reaction may be from −78° C. to 100° C.,−50° C. to 150° C., −25° C. to 100° C., 0° C. to 100° C., room temperature to 100° C. or 50° C. to 100° C.

### Effect of the Invention

According to the present disclosure, the use of 4-cyanobenzaldehyde in the step of synthesizing an indole skeleton with substituents introduced at the 2, 3, 4, and 6 positions offers two main advantages. First, it is relatively inexpensive and easy to procure, and second, due to the high crystallinity of the obtained compound, it allows for easy separation of the product through recrystallization. Consequently, the method for synthesizing rucaparib according to the present disclosure has superior advantages in terms of cost efficiency and yield compared to conventional manufacturing methods.

### BRIEF DESCRIPTION OF THE DRAWINGS

FIG. 1 is an NMR spectrum of 2-(4-cyanophenyl)-6-fluoro-4-methoxycarbonyl-indole-3-acetonitrile (Compound 3).

FIG. 2 is an NMR spectrum of N-desmethyl Rucaparib (Compound 2).

FIG. 3 is an NMR spectrum of rucaparib (Compound 1).

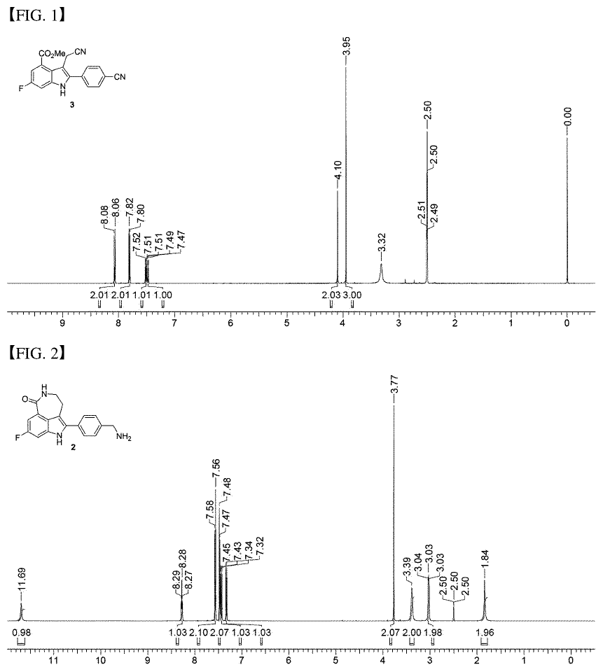

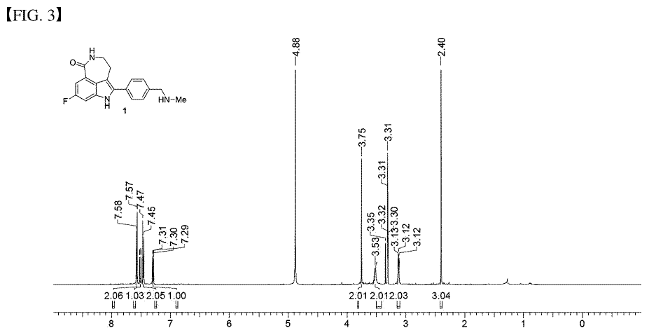

### BEST MODE FOR CARRYING OUT THE INVENTION

Hereinafter, the present disclosure will be described in more detail with reference to examples. However, the following examples are provided to help understand the present disclosure, and the scope of the present the present disclosure is not limited to the following examples.

### General Procedure

Unless otherwise stated, all reactions were performed in oven-dried glassware under an argon atmosphere. Unless otherwise indicated, all reactions were magnetically stirred, monitored by analytical thin layer chromatography (TLC) using a silica gel glass plate (0.25 mm) pre-coated with an F254 indicator, and visualized with UV light (254 nm). Flash column chromatography was performed using silica gel 60 (230 mesh to 400 mesh) with an indicated eluent. Commercial grade reagents were used without further purification. Unless otherwise stated, a yield refers to chromatographically and spectroscopically pure compound. ¹H NMR and ¹³C NMR spectra were recorded on 500 MHz and 125 MHz spectrometers, respectively. Tetramethylsilane (8TMS: 0.0 ppm) and residual NMR solvent (CDCl3 (δ_{H}: 7.26 ppm, dc: 77.16 ppm) or (CD₃)₂SO (δ_{H}: 2.50 ppm, dc: 39.52 ppm) were used as internal standards for ¹H NMR and ¹³C NMR spectra, respectively. A proton spectrum was expressed as 8 (proton position, multiplicity, coupling constant J, number of protons). Multiplicity was expressed as s (singlet), d (doublet), t (triplet), q (quartet), p (quintet), m (multiplet) and br (broad). A high-resolution mass spectrum (HRMS) was recorded on a quadrupole time-of-flight mass spectrometer (QTOF-MS) using electrospray ionization (ESI) as an ionization method.

Main reagents used are TiCl₄, NaBH₄, methanol (MeOH: for analysis, ACS grade, Carlo Erba Reagents) and the like.

### MODE FOR CARRYING OUT THE INVENTION

### Synthesis Example 1:2-(4-Cyanophenyl)-6-fluoro-4methoxycarbonyl-indole-3-acetonitrile (Compound 3)

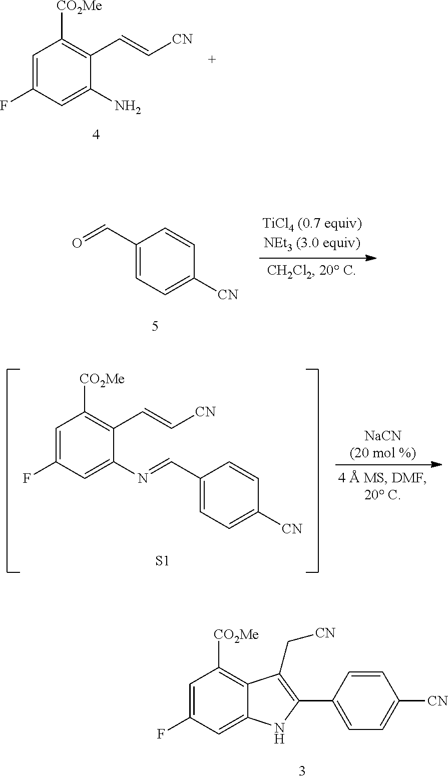 [SMILES: COC(=O)c1cc(F)cc(N)c1/C=C/C#N ; N#Cc1ccc(C=O)cc1 ; COC(=O)c1cc(F)cc(/N=C/c2ccc(C#N)cc2)c1/C=C/C#N ; "S1" ; COC(=O)c1cc(F)cc2[nH]c(-c3ccc(C#N)cc3)c(CC#N)c12]

Titanium tetrachloride (1.0 M dichloromethane solution, 7.0 mL, 7.0 mmol) was added to a dichloromethane (100 mL) solution of 2-Aminocinnamyl nitrile (Compound 4) (2.2 g, 10 mmol), aldehyde compound 5 (1.3 g, 10 mmol), and triethylamine (4.2 mL, 30 mmol), and while stirring the reaction mixture at 20° C., the progress of the reaction was observed by TLC and ¹H NMR analysis. After complete consumption of Compounds 4 and 5, distilled water (100 mL) was added dropwise, and the obtained mixture was extracted three times with dichloromethane (100 mL). The organic layer obtained was dried over MgSO₄, and concentrated to yield a mixture of imine compound S1, which was washed with hexane and used in the next reaction.

The mixture of imine compound S1 was dissolved in dimethylformamide (100 mL), and 4 Å molecular sieves (4.0 g) and sodium cyanide (98 mg, 2.0 mmol) were added, and while stirring the reaction mixture at 20° C., the progress of the reaction was observed by TLC. After complete consumption of compound S1, insoluble solids were filtered and removed, and the filtrate was washed with ethyl acetate. The obtained filtrate was concentrated, and dichloromethane was added dropwise to the reaction mixture to precipitate a white solid, which was filtered to yield compound 3 (2.7 g, 8.0 mmol, 80% yield over two steps).

¹H NMR (500 MHZ, DMSO-d₆) ò 8.07 (d, J=8.4 Hz, 2H), 7.81 (d, J=8.4 Hz, 2H), 7.52 (dd, J=8.9, 2.4 Hz, 1H), 7.48 (dd, J=10.1, 2.4 Hz, 1H), 4.10 (s, 2H), 3.95 (s, 3H); ¹³C {1H} NMR (125 MHZ, DMSO-d₆) ò 166.6 (d, J=2.7 Hz), 157.8 (d, J=237.9 Hz), 138.1 (d, J=3.6 Hz), 137.6 (d, J=12.7 Hz), 135.3, 132.9, 129.5, 124.0 (d, J=9.1 Hz), 121.3, 119.5, 118.6, 111.3 (d, J=26.3 Hz), 111.2, 102.5 (d, J=25.4 Hz), 102.4, 52.5, 15.8; 19F NMR (471 MHZ, DMSO-d₆) 8-119.9; HRMS (ESI-TOF) m/z: [M+Na]⁺ calcd for C₁₉H₁₂FN₃NaO₂ 356.0811; found: 356.0825.

### Synthesis Example 2: N-(tert-butoxycarbonyl)-2-(4-(N-tert-butoxycarbonylaminomethyl)phenyl)-6-fluoro-4-(methoxycarbonyl) tryptamine (Compound 6)

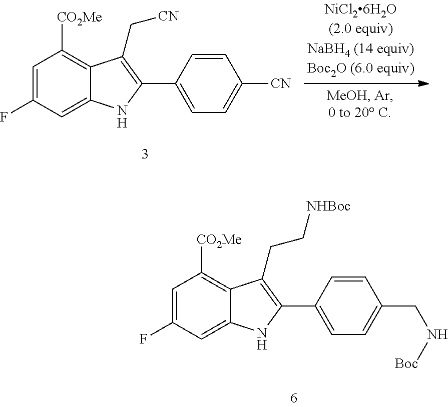 [SMILES: COC(=O)c1cc(F)cc2[nH]c(-c3ccc(C#N)cc3)c(CC#N)c12 ; COC(=O)c1cc(F)cc2[nH]c(-c3ccc(CNC(=O)OC(C)(C)C)cc3)c(CCNC(=O)OC(C)(C)C)c12]

Sodium borohydride (4.2 g, 112 mmol) was added to a methanol (80 mL) solution of Compound 3 (2.67 g, 8.0 mmol), di-tert-butyl dicarbonate (Boc20; 10.5 g, 48 mmol), and nickel chloride hexahydrate (NiCl2.6H2O; 3.8 g, 16 mmol) at 0° C., and while stirring the reaction mixture at 20° C., the progress of the reaction was observed by TLC. After complete consumption of compound 3, diethylenetriamine (17 mL, 160 mmol) was added dropwise and stirred for 30 minutes and then concentrated. To the mixture, a saturated NaHCO₃ aqueous solution (80 mL) was added dropwise, and the obtained mixture was extracted three times with ethyl acetate (80 mL). The organic layer obtained was dried over MgSO₄, concentrated, and was added dropwise to the reaction mixture to precipitate a white solid, which was filtered to yield compound 6 (3.47 g, 6.4 mmol, 80%).

¹H NMR (500 MHZ, DMSO-d₆) δ 11.70 (s, 1H), 7.59 (d, J=7.8 Hz, 2H), 7.49 (t, J=6.2 Hz, 1H), 7.34 (m, 3H), 7.25-7.20 (m, 1H), 6.90 (br, 1H), 4.21 (d, J=5.8 Hz, 2H), 3.93 (s, 3H), 3.00 (br, 4H), 1.42 (s, 9H), 1.36 (s, 9H); ¹³C {1H} NMR (125 MHz, DMSO-d₆) δ 167.4 (d, J=2.7 Hz), 157.1 (d, J=235.2 Hz), 155.9, 155.5, 140.1, 138.4, 137.2 (d, J=12.7 Hz), 130.5, 128.6, 127.1, 124.6 (d, J=9.1 Hz), 121.7, 109.1 (d, J=25.4 Hz), 109.0, 100.8 (d, J=24.5 Hz), 77.8, 77.3, 52.4, 43.1, 41.2, 28.3, 25.8; 19F NMR (471 MHz, DMSO-d₆) 8-122.3; HRMS (ESI-TOF) m/z: [M+Na]⁺ calcd for C₂₉H₃₆FN₃NaO₆ 564.2486; found: 564.2484.

### Synthesis Example 3: N-Desmethyl Rucaparib (Compound 2)

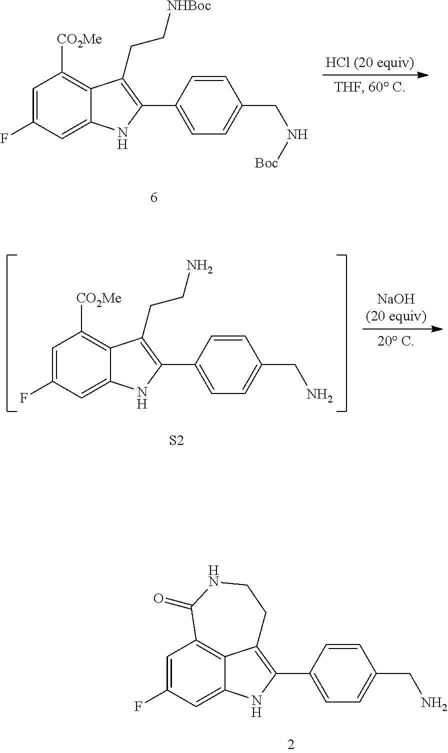

4 N hydrochloric acid aqueous solution (12.5 mL, 50 mmol) was added to a tetrahydrofuran (25 mL), and while stirring the reaction mixture at 60° C., the progress of the reaction was observed by TLC. After complete consumption of compound 6, the mixture of obtained compound S2 was cooled to 20° C., and 4 N sodium hydroxide aqueous solution (12.5 mL, 50 mmol) was added, and while stirring at the same temperature, the progress of the reaction was observed by TLC. After complete consumption of compound S2, the reaction mixture was concentrated to remove tetrahydrofuran. The precipitated solid from the remaining aqueous layer was filtered to yield an ivory solid, compound 2 (773 mg, 2.5 mmol, 100% yield over two steps).

¹H NMR (500 MHZ, DMSO-d₆) δ11.69 (br, 1H), 8.28 (t, J=5.7 Hz, 1H), 7.57 (d, J=8.1 Hz, 2H), 7.47 (d, J=8.2 Hz, 2H), 7.44 (dd, J=11.0, 2.3 Hz, 1H), 7.33 (dd, J=9.0, 2.4 Hz, 1H), 3.77 (s, 2H), 3.39 (br, 2H), 3.09-3.00 (m, 2H), 1.84 (br, 2H); ¹³C {1H} NMR (125 MHz, DMSO-d₆) δ 168.5 (d, J=1.8 Hz), 158.3 (d, J=234.3 Hz), 144.0, 136.7 (d, J=12.7 Hz), 135.5 (d, J=3.6 Hz), 129.6, 127.6, 127.4, 125.7 (d, J=9.1 Hz), 123.3, 111.4, 109.5 (d, J=25.4 Hz), 100.6 (d, J=25.4 Hz), 45.4, 41.9, 28.8; 19F NMR (471 MHZ, DMSO-d₆) 8-121.4; HRMS (ESI-TOF) m/z: [M+H]⁺ calcd for C₁₈H₁₇FN₃₀ 310.1356; found: 310.1353.

### Synthesis Example 4-1: Method for Preparing Rucaparib (Compound 1)

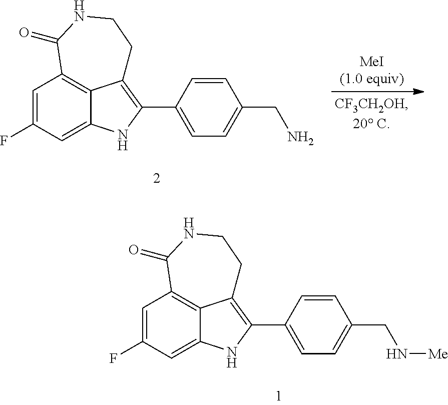 [SMILES: NCc1ccc(-c2[nH]c3cc(F)cc4c3c2CCNC4=O)cc1 ; CNCc1ccc(-c2[nH]c3cc(F)cc4c3c2CCNC4=O)cc1]

Methyl iodide (0.062 mL, 1.0 mmol) was added to a 2,2,2-trifluoroethanol (10 mL) solution of compound 2 (310 mg, 1.0 mmol), and while stirring the reaction mixture at 20° C., the progress of the reaction was observed by TLC. After 10 hours of the reaction, the reaction mixture was concentrated, and the product was purified by silica gel column chromatography using a solvent mixture of dichloromethane, methanol, and triethylamine (90:10:1) to yield a white solid, rucaparib 1 (110 mg, 0.34 mmol, 34%).

¹H NMR (500 MHZ, CD₃OD) δ 7.57 (d, J=8.2 Hz, 2H), 7.51 (dd, J=10.8, 2.3 Hz, 1H), 7.46 (d, J=8.2 Hz, 2H), 7.30 (dd, J=9.0, 2.4 Hz, 1H), 3.75 (s, 2H), 3.53 (br, 2H), 3.15-3.10 (m, 2H), 2.40 (s, 3H); ¹³C {1H} NMR (125 MHz, CD₃OD) § 172.6, 160.6 (d, J=235.2 Hz), 140.2, 138.6 (d, J=11.8 Hz), 137.3 (d, J=3.6 Hz), 132.3, 130.0, 129.2, 125.8 (d, J=9.1 Hz), 125.0, 112.9, 111.2 (d, J=26.3 Hz), 102.2 (d, J=26.3 Hz), 56.0, 43.8, 35.6, 30.0; 19F NMR (471 MHz, CD₃OD) δ-123.4; HRMS (ESI-TOF) m/z: [M+H]⁺ calcd for C₁₉H₁₉FN₃₀ 324.1507; found: 324.1510.

### Synthesis Example 4-2: Method for Preparing Rucaparib

### (Compound 1)

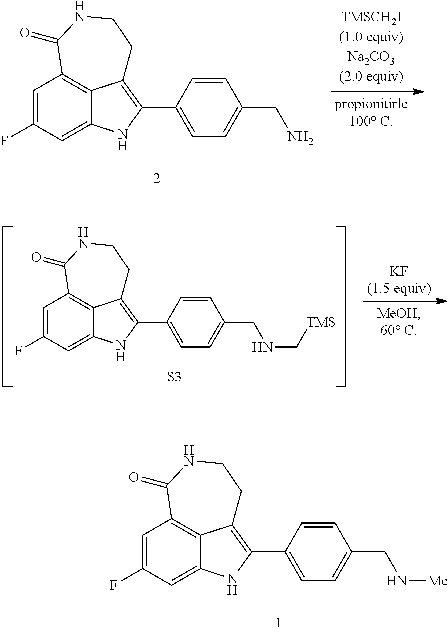

(Iodomethyl)trimethylsilane (0.15 mL, 1.0 mmol) was added to a propionitrile (10 mL) solution of compound 2 (310 mg, 1.0 mmol) and sodium carbonate (210 mg, 2.0 mmol) in propionitrile (10 mL), and while stirring the reaction mixture at 60° C., the progress of the reaction was observed by TLC. After complete consumption of compound 2, the insoluble solids were filtered and removed, and the filtrate was washed with ethyl acetate. The obtained filtrate was concentrate to yield a mixture of compound S3, which was used directly in the next reaction without further purification.

The mixture of compound S3 was dissolved in methanol (10 mL), and potassium fluoride (87 mg, 1.5 mmol) was added, and while stirring the reaction mixture at 60° C., the progress of the reaction was observed by TLC. After complete consumption of compound S3, the reaction mixture was concentrated, and the product was purified by silica gel column chromatography using a solvent mixture of dichloromethane, methanol, and triethylamine (90:10:1) to yield a white solid, rucaparib 1 (184 mg, 0.57 mmol, 57% yield over two steps).

¹H NMR (500 MHZ, CD₃OD) & 7.57 (d, J=8.2 Hz, 2H), 7.51 (dd, J=10.8, 2.3 Hz, 1H), 7.46 (d, J=8.2 Hz, 2H), 7.30 (dd, J=9.0, 2.4 Hz, 1H), 3.75 (s, 2H), 3.53 (br, 2H), 3.15-3.10 (m, 2H), 2.40 (s, 3H); ¹³C {1H} NMR (125 MHz, CD₃OD) & 172.6, 160.6 (d, J=235.2 Hz), 140.2, 138.6 (d, J=11.8 Hz), 137.3 (d, J=3.6 Hz), 132.3, 130.0, 129.2, 125.8 (d, J=9.1 Hz), 125.0, 112.9, 111.2 (d, J=26.3 Hz), 102.2 (d, J=26.3 Hz), 56.0, 43.8, 35.6, 30.0; ¹⁹F NMR (471 MHz, CD₃OD) δ-123.4; HRMS (ESI-TOF) m/z: [M+H]⁺ calcd for C₁₉H₁₉FN₃₀ 324.1507; found: 324.1510.

1. A method for preparing a compound of Chemical Formula (3), the method comprising:
reacting a compound of the following Chemical Formula (1) and a compound of the following Chemical Formula (2); and
converting the result into a compound of the following Chemical Formula (3) in the presence of a catalyst: 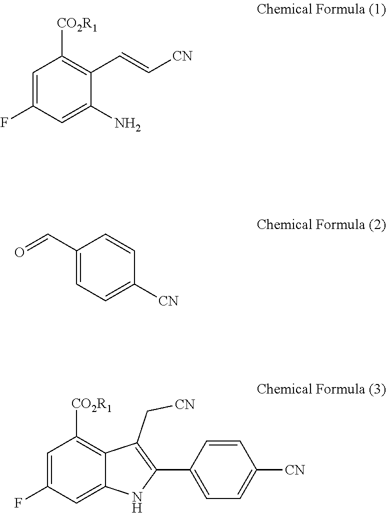
herein,
R₁ is a linear or branched C₁-C₅ alkyl.

2. The method of claim 1, wherein the step of converting the result into a compound of Chemical Formula (3) is performed in the presence of a dehydrating agent.

3. The method of claim 2, wherein the dehydrating agent is at least one compound selected from the group consisting of TiCl₄, MgSO₄ and Na₂SO₄, or a molecular sieve.

4. The method of claim 1, wherein the step of converting the result into a compound of Chemical Formula (3) is performed using an azeotropic distillation.

5. The method of claim 1, wherein the catalyst is MCN or N-heterocyclic carbene:
herein, M is an alkali metal or NR₄⁺; and
R is H or a linear or branched C₁-C₅alkyl.

6. The method of claim 5, wherein the N-heterocyclic carbene is selected from the group consisting of imidazolium, triazolium and thiazolium.

7. A method for preparing rucaparib represented by the following compound 1 includes:
(a) reacting a compound of the following Chemical Formula (1) and a compound of the following Chemical Formula (2), and converting the result into a compound of the following Chemical Formula (3) in the presence of a catalyst;
(b) performing a reduction reaction of the compound of Chemical Formula (3) and protecting the obtained primary amine with P₁ and P₂, thereby converting it into a compound of Chemical Formula (4);
(c) performing a deprotection reaction on the compound of Chemical Formula (4), followed by or simultaneously with a lactam ring formation reaction to obtain a compound of Chemical Formula (5); and
(d) performing a monomethylation reaction on the primary amine group of the compound of Chemical Formula (5) to produce Compound 1. 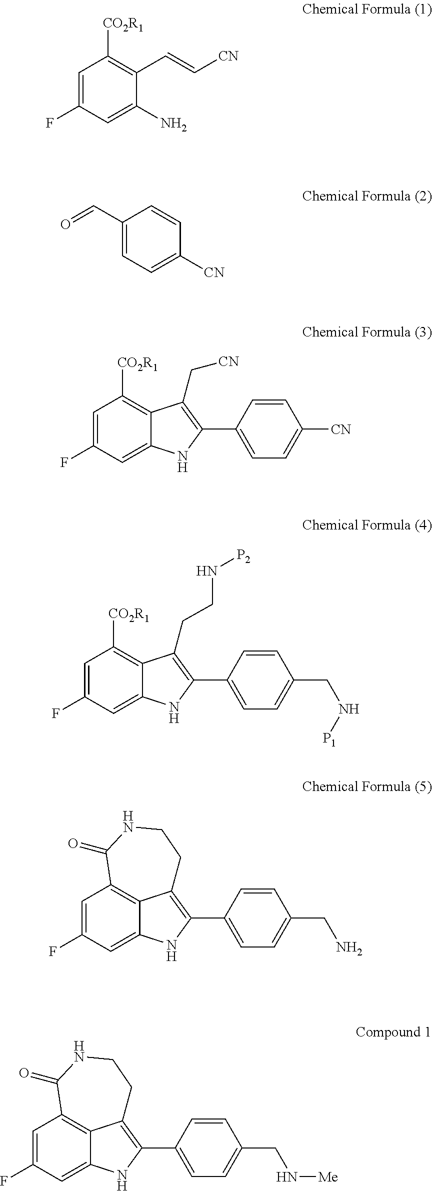
herein,
R₁ is a linear or branched C₁-C₅ alkyl;
P₁ and P₂ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

8. The method of claim 7, wherein the step (a) is performed in the presence of a dehydrating agent.

9. The method of claim 8, wherein the dehydrating agent is at least one compound selected from the group consisting of TiCl₄, MgSO₄ and Na₂SO₄, or a molecular sieve.

10. The method of claim 7, wherein the step (a) is performed using an azeotropic distillation.

11. The method of claim 7, wherein the catalyst used in step (a) is MCN or N-heterocyclic carbene:
herein, M is an alkali metal or NR₄⁺; and
R is H or a linear or branched C₁-C₅ alkyl.

12. The method of claim 7, wherein the reduction reaction in step (b) is performed in the presence of nickel boride.

13. The method of claim 7, wherein the reduction reaction in step (b) is a hydrogenation reaction performed in the presence of a metal catalyst selected from the group consisting of Ni, Pd, and Pt.

14. The method of claim 7, wherein the reduction reaction in step (b) is performed in the presence of a metal catalyst selected from the group consisting of Ni, Zn, Fe and Co, and a silane compound.

15. The method of claim 7, wherein the reduction reaction in step (b) is performed in the presence of a metal hydride selected from the group consisting of DIBAL-H, L-selectride, NaBH₄ and borane.

16. The method of claim 7, the deprotection reaction in step (c) is performed under acidic conditions.

17. The method of claim 7, wherein the lactam ring formation reaction in step (c) is performed under basic conditions.

18. The method of claim 7, wherein the monomethylation reaction in step (d) of the primary amine in the compound of Chemical Formula (5) is performed in a one-step reaction using Me-X,
herein, X is a halogen or a leaving group.

19. The method of claim 18, wherein Me-X is iodomethane.

20. The method of claim 7, the monomethylation reaction in step (d) is performed by reacting the primary amine of the compound of Chemical Formula (5) with CHXYZ to introduce one carbon atom, followed by replacing Y and Z with hydrogen in a multi-step process:
herein, X is a halogen or a corresponding leaving group, and Y and Z are substituents that can be replaced with hydrogen.

21. The method of claim 7, the monomethylation reaction in step (d) is performed by reacting the primary amine the compound of Chemical Formula (5) with CH₂XY to introduce a CH₂Y group, followed by replacing Y with hydrogen in a multi-step process:
herein, X is a halogen or a corresponding leaving group, and Y is a substituent that can be replaced with hydrogen.

22. The method of claim 21, wherein CH₂XY is (iodomethyl)trimethylsilane.

23. A compound of the following Chemical Formula (3) used in preparation of rucaparib: 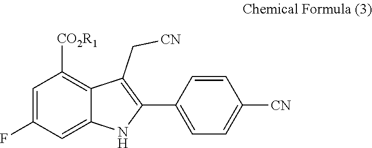
herein,
R₁ is a linear or branched C₁-C₅alkyl.
